(19) United States
(12) Patent Application Publication
(10) Pub. No.: US 2017/0157382 A1
(43) Pub. Date: Jun. 8, 2017

(54) TATTOO MACHINE FLOATING COLLET APPARATUS AND SYSTEM

(71) Applicant: FK IRONS, INC., Doral, FL (US)

(72) Inventor: GASTON SICILIANO, Miami, FL (US)

(73) Assignee: FK IRONS, INC., Doral, FL (US)

(21) Appl. No.: 14/963,202

(22) Filed: Dec. 8, 2015

(51) Int. Cl. A61M 37/00; B23B 31/02; B23B 31/20

(52) U.S. Cl. CPC A61M 37/0076; B23B 31/20; B23B 31/028

(57) ABSTRACT

The present invention discloses a tattoo machine floating collet apparatus and system comprising a vise receiver, a vise knob, and a vise collet that are configured to receive a tattoo machine grip backstem. These components are configured as follows: vise collet is mechanically captured between the vise receiver and the vise knob such that as the vise knob is tightened onto the vise receiver the collet tightens around an inserted tattoo machine grip backstem. The invention can be used to provide a quick, easy, and sanitary apparatus for tattoo artists to use multiple grips and needle cartridges with the same tattoo machine.

### CROSS-REFERENCE TO RELATED APPLICATIONS

None.

### STATEMENT REGARDING FEDERALLY SPONSORED-RESEARCH OR DEVELOPMENT

None.

### FIELD OF THE INVENTION

The invention relates generally to the field of mechanical apparatuses, and more particularly, to a tattoo machine with a floating collet apparatus and system configured to accept multiple tattoo machine grip styles.

### BACKGROUND

Tattoo machines generally use electric coils to drive a spring-loaded reciprocating needle structure in order to etch skin. This type of tattoo etching machine is heavy and cumbersome for the operator (the tattoo artist), is prone to overheating, and is inherently noisy during operation. Due to the nature of the prior designs, with exposed coils and electrical connections, it has not been possible to properly clean, much less sterilize or autoclave, the tattoo machine itself. This creates an inherent health and safety issue for the customer as well as for the artist.

Some solutions in the art have attempted to address the problem addressed above by separating the grip portion, which accepts the reciprocating needle, from the machine portion, which converts electrical energy into a reciprocating motion. However, this falls short in that many grip manufacturers create specific grip-machine interfaces that make it necessary to use their grip-machine combination. Tattoo machine users, being artists, prefer to have the flexibility to use various needle-grip-machine combinations in much the same way a painter utilizes different brushes.

Other solutions in this area are deficient in that: the components susceptible to be in contact with human fluids cannot be properly sterilized; the stroke of the reciprocating needle is not adjustable; the reciprocating needle driver has no give; the machines are not cross-compatible with other needle, grip, and cartridge systems; and the machines are cumbersome, noisy, and heavy.

It would, therefore, be desirable to have a tattoo machine with adjustable stroke, that is compatible with multiple tattoo needle cartridge systems and grip styles, with a self-aligning and sterilizable drive bar that has adjustable give, and is lightweight and virtually vibration free.

While certain aspects of conventional technologies have been discussed to facilitate disclosure of the invention, Applicant in no way disclaims these technical aspects, and it is contemplated that the claimed invention may encompass one or more of the conventional technical aspects discussed herein.

In this specification where a document, act, or item of knowledge is referred to or discussed, this reference or discussion is not an admission that the document, act, or item of knowledge or any combination thereof was at the priority date, publicly available, known to the public, part of common general knowledge, or otherwise constitutes prior art under the applicable statutory provision; or is known to be relevant to an attempt to solve any problem with which this specification is concerned.

### SUMMARY

A preferred embodiment of the present invention is directed to a tattoo machine floating collet apparatus. The apparatus comprises a vise receiver, a vise receiver knob, and a vise collet. In this embodiment, the components of the apparatus are configured to accept a tattoo machine grip backstem. The components are arranged so that the vise collet is captured between the vise receiver and the vise knob. In use, the backstem portion of a tattoo machine grip is inserted into the vise receiver through the vise knob and vise collet. As the vise knob is tighten down onto the vise receiver, the vise collet tightens down on the backstem of the tattoo machine grip and holds it in place.

Another embodiment of the present invention is directed to an apparatus that satisfies the need for a tattoo machine that is designed to be compatible with multiple grips and grip styles. This embodiment comprises a drive bar, a dampener, a slider, a stroke module, a vise receiver, and a housing.

Further embodiments may also comprise a vise receiver collet and knob, a removable motor assembly, and adjustable stroke module stroke distance.

Yet another embodiment may comprise a collet vise system for accepting tattoo machine cartridge grips. Embodiments of such a system may comprise a tattoo machine apparatus, a collet apparatus, and a vise knob apparatus.

Certain embodiments of the present invention may be used for, but not limited to, the etching of human skin for tattooing. It is contemplated, however, that the use of certain embodiments of the present invention in other applications beyond traditional tattoo use, is within the scope of the present invention. For example, the invention may be used for cosmetic tattooing, use in applications other than on humans, or for other medical or industrial uses.

Embodiments of the present invention may include components manufactured from various materials based upon the contemplated use. For embodiments that are contemplated for human use, materials that are durable, autoclaveable, and sanitary are contemplated to be within the scope of the present invention. By way of example and not limitation, materials may be stainless steel, anodized aluminum, or polycarbonates.

The present invention may address one or more of the problems and deficiencies of the prior art discussed above. However, it is contemplated that the invention may prove useful in addressing other problems and deficiencies in a number of technical areas. Therefore the claimed invention should not necessarily be construed as limited to addressing any of the particular problems or deficiencies discussed herein.

### BRIEF DESCRIPTION OF THE DRAWINGS

These and other features, aspects, and advantages of the present invention will become better understood with regard to the following description, appended claims, and accompanying drawings where:

FIG. 1 is an isometric view showing an embodiment of the invention in use in conjunction with a tattoo machine and a tattoo machine grip;

FIG. 2 is a composite view showing a top view and a cross-sectional view of an embodiment of the invention in use in conjunction with a tattoo machine and a tattoo machine grip;

FIG. 3 is a composite view showing an isometric view and an exploded isometric view of an embodiment of the invention showing a tattoo machine floating collet apparatus;

FIG. 4 is a composite view showing an isometric view and an exploded isometric view of an embodiment of the invention showing a collet vise system;

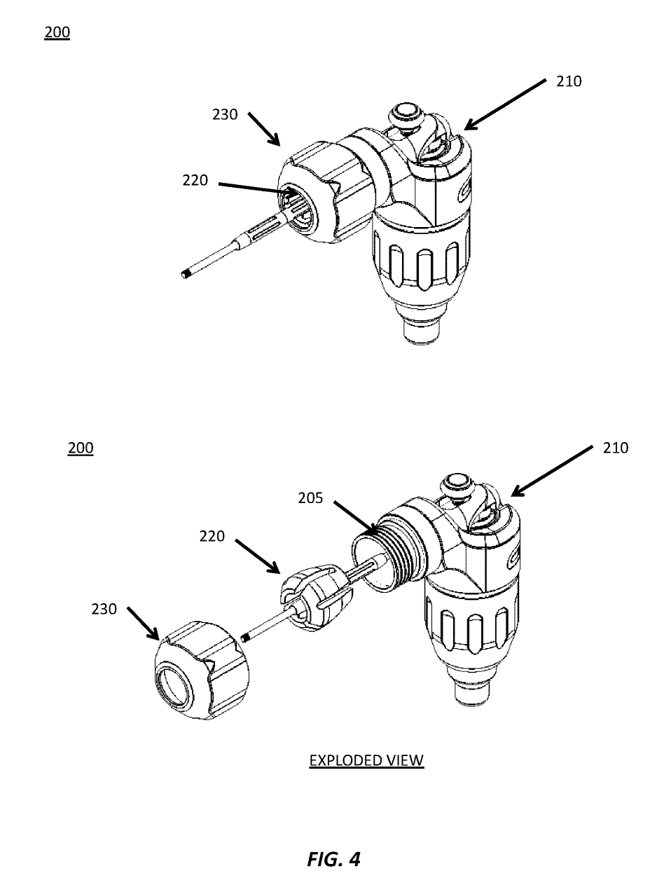

FIG. 5 is an isometric view in accordance with an embodiment of the present invention showing a tattoo machine with floating vise collet apparatus;

FIG. 6 is a composite view showing orthographic projections in accordance with an embodiment of the present invention showing a tattoo machine with floating vise collet apparatus;

FIG. 7 is an exploded view in accordance with an embodiment of the present invention showing a tattoo machine with floating vise collet apparatus; and

FIG. 8 is a composite view showing isometric and cross-sectional views of embodiments of the vise receiver knob and the vise collet in accordance with embodiments of the present invention.

### DESCRIPTION

In the Summary above, in the Description and appended Claims below, and in the accompanying drawings, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

The term “comprises” and grammatical equivalents thereof are used herein to mean that other components, structures, steps, etc. are optionally present. For example, an article “comprising” (or “which comprises”) components A, B, and C can consist of (i.e., contain only) components A, B, and C, or van contain not only components A, B, and C, but also one or more other components or structures.

The term “at least” followed by a number is used herein to denote the start of a range beginning with that number (which may be a range having an upper limit or no upper limit, depending on the variable being defined). For example, “at least 1” means 1 more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40% means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm.

The term “mechanical features” is used herein to mean features of a component, mechanical or geometric, which have a functional purpose of attaching or linking that component to one or more other components with compatible or corresponding mechanical features. An example of a mechanical feature is a slot in a component, where said slot is designed to accept a tab from another component and the union of the slot and tab from the two components effectively links, attaches, fixes, and/or locks the components together. The term “mechanical features” refers to, but is not limited to: hooks, hook and loop fasteners, slot and tabs, all male and female fasteners, screws, bolts, nuts, holes that have been tapped, latches, pins, etc.

While the specification will conclude with claims defining the features of embodiments of the invention that are regarded as novel, it is believed that the invention will be better understood from a consideration of the following description in conjunction with the figures, in which like reference numerals are carried forward.

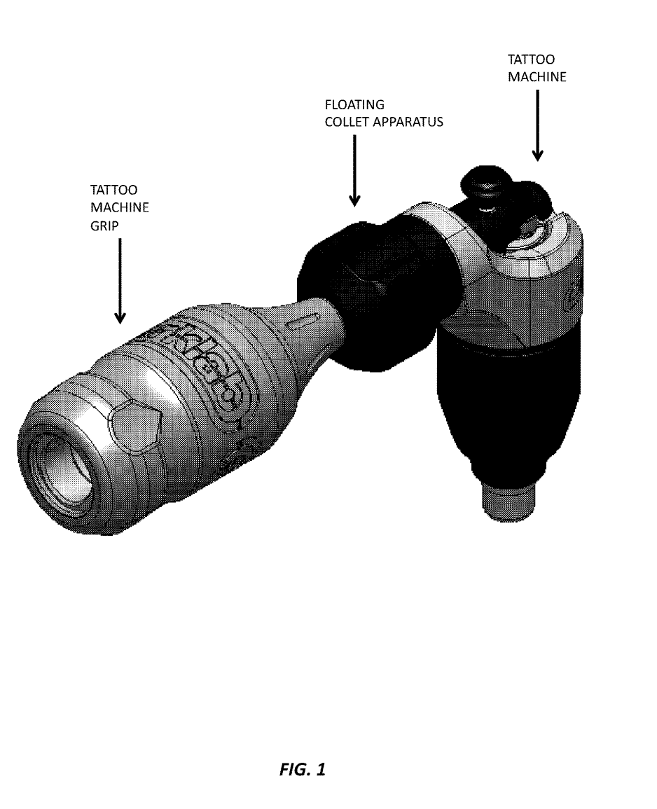

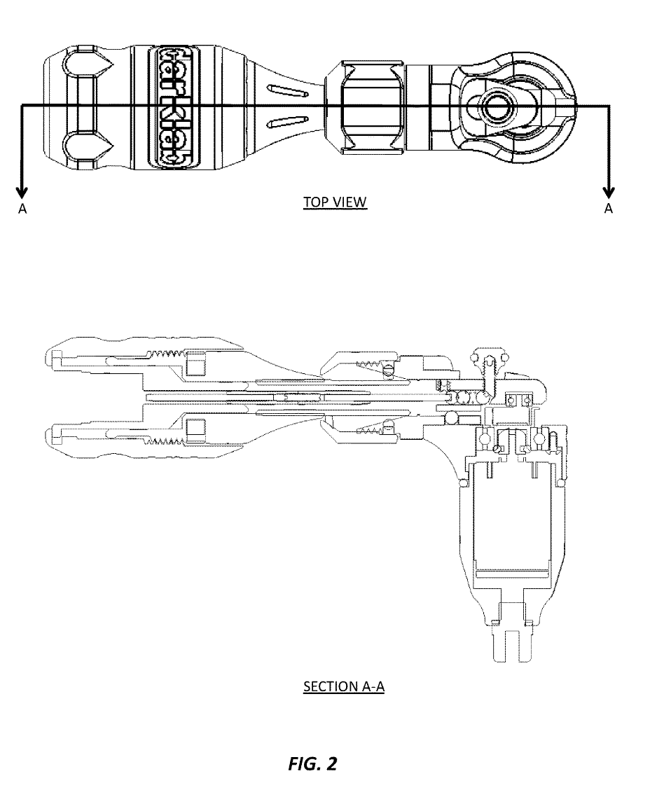

For reference, and to provide structure and context for the description of the various embodiments of the present invention, we first refer to FIGS. 1 and 2. FIG. 1 shows an isometric view of an assembly incorporating an embodiment of the present invention along with other structural components for reference. FIG. 2 shows that same assembly in a top view as well as a longitudinal cross-sectional view. Tattoo machine grips come in many different styles, and from many different manufacturers. Likewise, the tattoo machines themselves may come in many different styles and from different manufacturers. What we show in FIGS. 1 and 2 is how an embodiment of the present invention may function in a way that couples the tattoo machine grip to the tattoo machine.

Tattoo Machine Floating Collet Apparatus 100

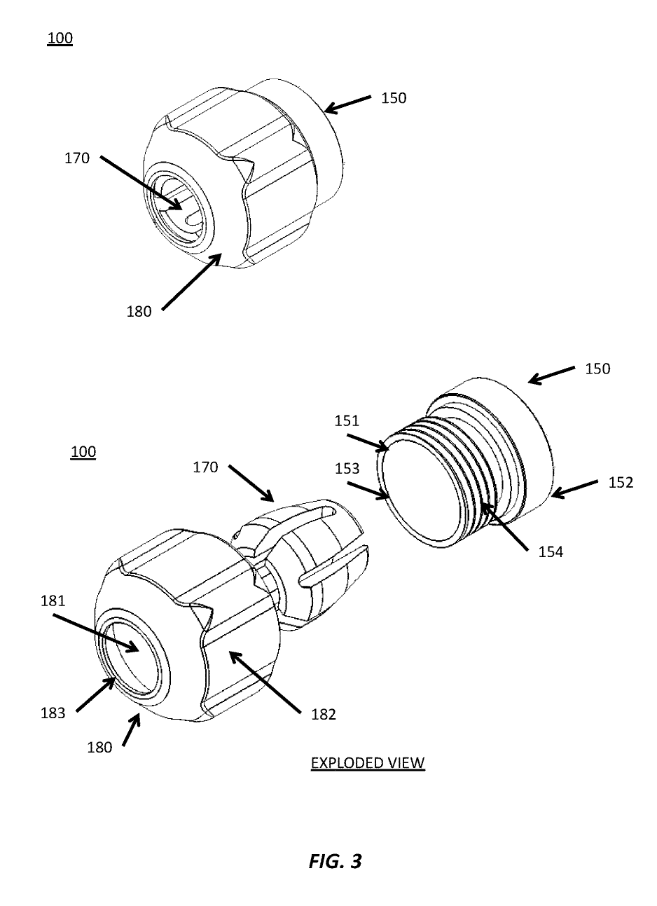

Referring to the figures in general, and to FIG. 3 in particular, a preferred embodiment of the present invention in the form of a tattoo machine floating collet apparatus 100 is disclosed here. In the preferred embodiment, the apparatus comprises the following components and structures: a vise receiver 150, a vise receiver knob 180, and a vise collet 170. The components are related as follows: the vise collet 170 is configured to be at least partially inserted into the vise receiver 150, the vise receiver knob 180 is configured to mechanically attach to the vise receiver 150 and capture the vise collet 170 with respect to the vise receiver 150.

The Vise Receiver 150

We discuss now the vise receiver component of the apparatus. FIG. 3 shows a vise receiver 150 configured to receive a tattoo machine grip backstem. The vise receiver has a first end 151, a second end 152, and a body wall 153 extending between the first end and second end. This body wall defines an inner surface and an outer surface. At least a portion of the outer surface extending from the first end towards the second end has an externally threaded portion 154.

Further embodiments including the vise receiver may further comprise a vise receiver with a straight, variable, or conical inner surface. Yet further embodiments may include the vise receiver as comprising a portion of a tattoo machine.

The Vise Receiver Knob 180

We discuss now the vise receiver knob component of the apparatus. FIG. 3 shows a vise receiver knob 180 having an inner surface 181 that is at least partially threaded so as to mechanically engage with the vise receiver externally threaded portion, an outer surface 182, and an aperture 183 configured to receive a tattoo machine grip backstem.

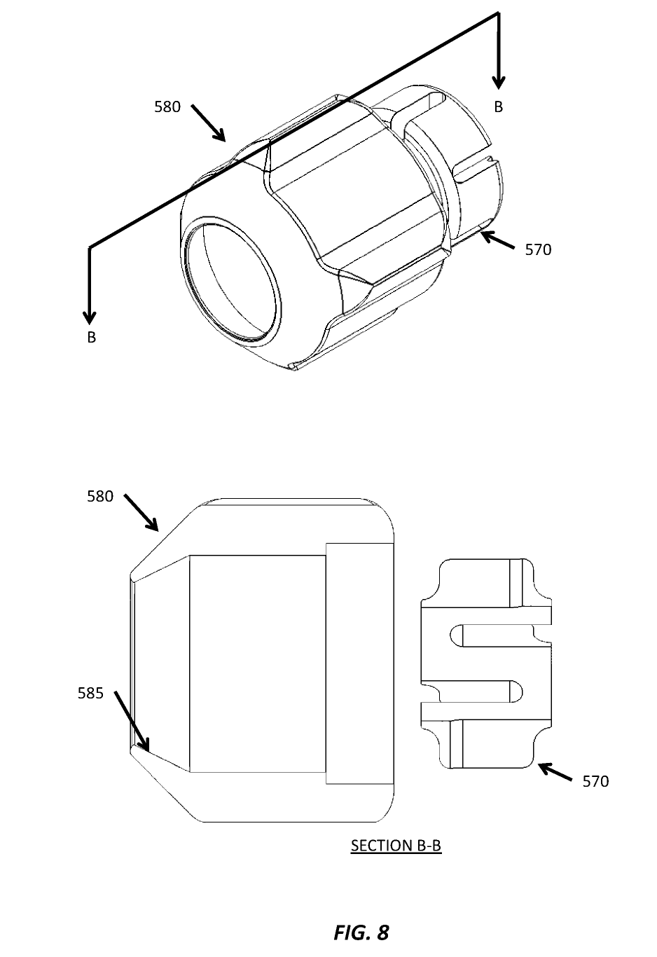

Further embodiments may include a vise receiver knob 580 where the inner surface of the vise receiver knob includes a conical taper 585, as shown in FIG. 8.

Other embodiments of the vise receiver knob component may include features on the outer surface to facilitate gripping. Such features may include: one or more protrusions, one or more ridges, one or more knurled areas, or other gripping means. FIG. 3. shows an embodiment where the outer surface 182 has multiple radially disposed protrusions for gripping the vise receiver knob.

The Vise Collet 170

We discuss now the vise collet component of the apparatus. FIG. 3 shows a vise collet 170 component that is configured to accept a tattoo machine grip backstem and be coaxially inserted into the vise receiver first end such that when the vise receiver knob is threaded onto the external threads of the vise receiver first end, the vise receiver knob engages with the vise collet, thereby causing the vise collet to tighten onto the tattoo machine grip backstem.

Further embodiments of the apparatus may include a vise collet that is substantially conical (as shown in FIG. 3). In this way, as the vise collet is pushed into the vise receiver by the vise receiver knob, the walls of the vise receiver and the tapered (conical) walls of the vise collet interact, thereby causing a reduction in the inner diameter of the central bore of the vise collet.

Yet another embodiment of the vise collet 570 is shown in FIG. 8. In this embodiment, the vise collet is substantially cylindrical. Conical, or sloping, features in the vise receiver knob would interact with the substantially straight walls of this embodiment of the vise collet in order to cause a reduction in the inner diameter of the central bore of the vise collet.

Yet another embodiment of the vise collet includes features on the inner surface (within the central bore) that interact, or “key” with a tattoo machine grip backstem in order to counteract rotational movement of the tattoo machine grip backstem when inserted into the apparatus.

Collet Vise System Embodiment 200

A further embodiment of the present invention includes a collet vise system 200 for accepting tattoo machine grips. In this embodiment, the system is comprised of a tattoo machine apparatus 210, a collet apparatus 220, and a vise knob apparatus 230.

The tattoo machine apparatus 210 is a component having at least an externally threaded portion 205 substantially similar to the vise receiver 150 disclosed here. Some versions of the tattoo machine apparatus are configured to accept a screw-on style adjustable grip with internal threading.

The collet apparatus 220 is a component having features substantially similar to the vise collet 170 disclosed here. The collet apparatus 230 has at least an outer surface, an inner surface with an adjustable inner diameter configured to accept a standard backstem style grip tube. In operation, this collet apparatus is at least partially inserted into the tattoo machine apparatus externally threaded vise receiver portion.

The vise knob apparatus 230 is a component having features substantially similar to the vise receiver knob 180 disclosed here. The vise knob apparatus 230 has at least a first end, an outer surface, a second end distal to the first end, a central bore extending from the first end to the second end defining an inner surface, where a portion of the inner surface extending inward from the second end is threaded such that the thread is configured to mate with the thread of the tattoo machine apparatus externally threaded vise receiver, and a portion of the inner surface extending inward from the first end has a one or more internal bearing surface configured to contact with the collet apparatus when the inner surface threads are engaged with the tattoo machine apparatus vise receiver external threads.

The collet vise system embodiment described here can be used, among other uses, to accept tattoo machine grips. With this system embodiment, a tattoo artist, for example, can quickly and easily swap out different tattoo machine grips while still using the same tattoo machine.

It is understood that the variations and options disclosed above with respect to the components of the apparatus embodiments, are also applicable as variations and options with respect to the components for the system embodiments.

Tattoo Machine Floating Vise Collet Apparatus Embodiment 1000

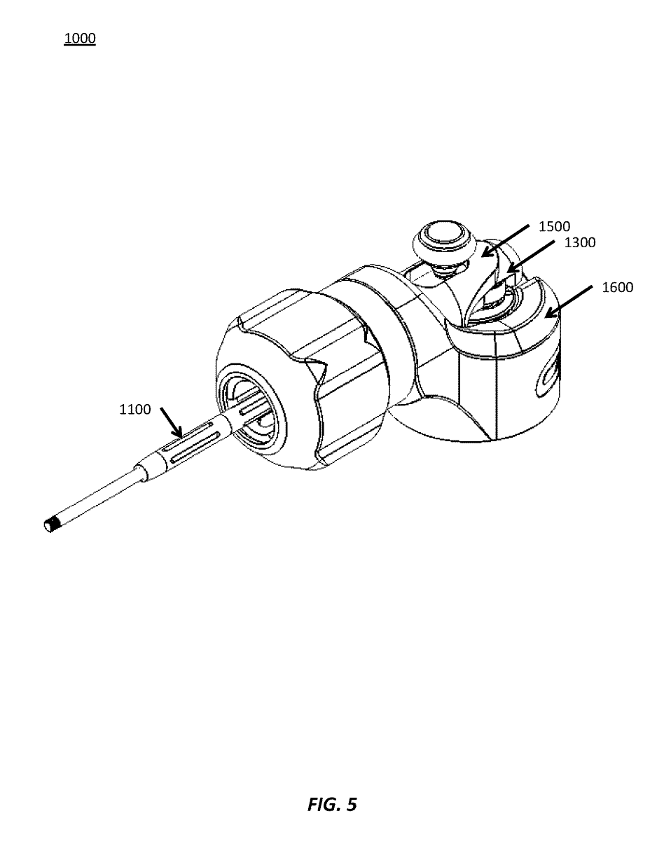

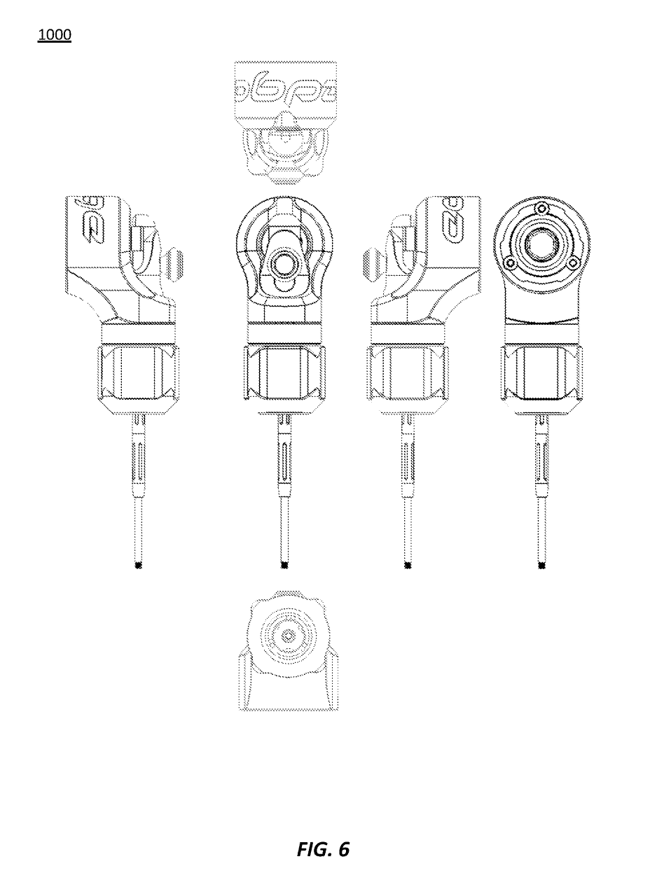

Referring to the figures in general, and to FIGS. 5, 6 and 7 in particular, an embodiment of the present invention is in the form of an apparatus 1000. The apparatus 1000 is comprised of a drive bar 110, a dampener 120, a slider 130, a stroke module 140, a vise receiver 150, and a machine housing 160.

The drive bar 110 is a push rod that mechanically couples the motion from an attached motor or drive mechanism with an attached needle mechanism in order to translate the motor's motion into the needle's reciprocating motion. The drive bar 110 has a first end, a distal second end, and a body defining a central longitudinal axis extending between the first end and the second end. The drive bar 110 is made of a sufficiently rigid material to not buckle under ordinary loads. Materials that are easily sterilized and antimicrobial are preferred.

The dampener 120 is a compressible component capable of absorbing, partially absorbing, or completely translating an applied longitudinal load. Embodiments of the dampener 120 may be accomplished using a coil spring, a compressible closed cell foam, or other material or assembly of materials that are capable of performing the function of absorbing, partially absorbing, or completely translating a longitudinal load. FIG. 10 illustrates a preferred embodiment of the damper 120 as an assembly of two ball bearings and a spring, where each ball bearing is in contact with one end of the spring and loads applied to the ball bearings are translated into the spring element. Embodiments of the damper 120 have a first surface and a second surface, where the first surface is in contact with the drive bar second end.

The slider 130 couples the stroke module 140 to the dampener 120, which is coupled to the drive bar 110. The slider 130 has a body portion with a central longitudinal axis, a first end, and a second end, where a section of the body portion at the second end has a first bearing surface, a section of the body portion at the first end has a blind bore coaxial with the central longitudinal axis extending from the first end into the body portion but not completely through the body portion so that the blind bore forms an opening in the body portion with a substantially cylindrical wall and a second bearing surface orthogonal to the central longitudinal axis and distal from the body portion first end, and the first bearing surface is in contact with the dampener 120 second surface. Although it is contemplated that the slider may be manufactured from various materials, a preferred embodiment of the present invention comprises a slider that is made of an acetal resin containing PTFE that will reduce friction and ensure the overall smooth operation and low vibration of the apparatus.

The stroke module 140 couples a motor to the slider 130. The stroke module has a cam portion and a receptacle portion. The receptacle portion is configured to be operably communicative with a motor such that motion from the motor is translated through the receptacle portion to the cam portion. In other words, as the receptacle portion is caused to rotate due to a motor, the cam portion likewise rotates. The stroke module 140 is assembled in relation to the slider 130 so that the cam is slidingly communicative with the slider first bearing surface.

The vise receiver 150 is configured to receive a tattoo needle cartridge. The vise receiver has a first end, a second end, and a body wall defining a one or more inner surface and a one or more outer surface extending between the first end and second end that has a central axis and varying cross-section, where the first end has an externally threaded portion and the second end is configured to receive the slider first end, at least a portion of the slider body. The vise receiver is further configured to allow the drive bar first end and at least a portion of the drive bar body to extend through the vise receiver and past the vise receiver first end. The vise receiver acts to constrain the slider to move only longitudinally along its central axis. The vise receiver further acts to accept standard screw-on adjustable grips.

The various components are constrained in relation to each other by a machine housing 160. The machine housing 160 has a first opening, a second opening, and a body. The first opening is configured to fixedly attach and constrain the vise receiver in translational and rotational relation to the machine housing. The second opening is configured to fixedly attach and constrain the stroke module in translational and rotational relation to the machine housing, but allowing the cam portion and receptacle portion of the stroke module to operate freely. The machine housing is further configured to maintain operable communication between the stroke module, the slider, and the vise receiver.

In this way, rotational motion of the receptacle portion is translated into rotational motion of the cam portion. The rotational motion of the cam portion is translated into longitudinal motion of the slider. Since the slider is longitudinally communicative with the drive bar, via the damper, the rotational motion of the receptacle portion is translated into reciprocating longitudinal motion of the drive bar. Furthermore, the second end of the drive bar is mechanically coupled to the dampener 120 such that it is free to pivot in relation to the central axis, but still reciprocate back and forth. This may be accomplished, for example, by making the drive bar second end terminate in a sphere. In this way, the pivoting drive bar self-aligns with the plungers of an attached needle cartridge, thus ensuring perfect rectilinear motion.

Variations on this embodiment are contemplated to be within the scope of the invention. For example, the dampener and the slider may be eliminated so that the drive bar is connected directly to the stroke module.

Other embodiments of the present invention may also comprise a vise receiver collet 170 and a vise receiver knob 180. In such an embodiment, the vise receiver collet is coaxially inserted into the vise receiver first end and the vise receiver knob is threaded onto the external threads of the vise receiver first end such that, as the vise receiver knob is rotated so as to tighten the vise receiver knob onto the vise receiver, the vise receiver knob pushes the vise receiver collet further into the vise receiver first end, thereby reducing the inner diameter of the vise receiver collet. In this way, the apparatus may be compatible with back stem style adjustable grips. In order to insert a standard back stem style grip, partially unscrew the vise receiver knob enough to loosen it but not fully remove it. Gently guide the back stem of the grip over the drive bar and insert it into the opening of the vise receiver knob, passing through the opening of the vise receiver collet. Once the back stem of the grip is inserted the desired distance, tighten the vise receiver knob in order to secure the grip in place. Embodiments of the invention contemplate the presence of ridges, or other protrusions, on the inside surface of the vise receiver collet, that are configured to interface with grooves in the back stem of a grip so as to counter or resist against any rotation of the back stem tube in the vise receiver collet.

Another embodiment of the present invention may further comprise a motor assembly. The motor assembly is comprised of a motor, an electrical connector, and a housing. The housing protects and encases the motor body, but allows the motor shaft and electrical connector to protrude from the housing. The motor assembly housing further has mechanical features that allow it to be removably attached to the machine housing second opening ensuring that the motor shaft and the stroke module receptacle portion are operatively communicative.

Yet another embodiment of the present invention may further comprise a stroke module where the stroke distance, the longitudinal displacement of the cam portion, is adjustable.

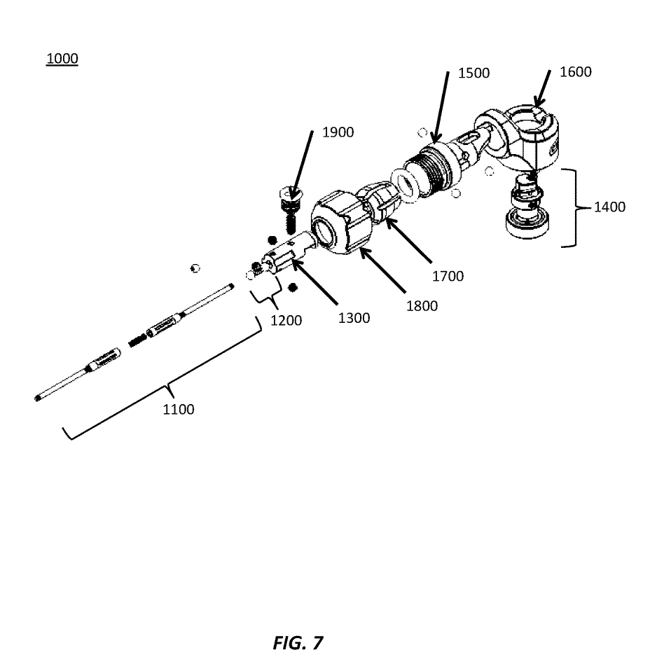

Yet another embodiment of the present invention may further comprise a dampener where the dampener resistance is adjustable. By way of illustration and not limitation, FIG. 7 shows a resistance knob 1900 that, when tightened, push on a ball bearing causing the dampener spring to compress, thereby stiffening the dampener. When the resistance knob is retracted, the spring can operate freely, thereby allowing for more “give” in the system.

Yet another embodiment of the present invention contemplates a two-part drive bar, where the drive bar body is separable by mechanical means into a first portion where the first end is removable from the system, and a second portion where the second end remains operably connected to the system. This two-part drive bar system allows for the portion of the drive bar that is in communication with the needle cartridge to be removed for sterilization. Additionally, this two-part drive bar allows for the ability to interchange the configuration of the drive bar first end in order to suit the requirements of a variety of grip styles.

An embodiment of the present invention in the form of a collet vise system for accepting tattoo machine cartridge grips comprises a tattoo machine apparatus, a collet apparatus, and a vise knob apparatus.

In this system embodiment, the tattoo machine apparatus has an externally threaded vise receiver, where said externally threaded vise receiver is substantially cylindrical with an internal bore having an internal bore diameter, and is configured to accept a screw-on style adjustable cartridge grip with internal threading.

The collet apparatus has a substantially conical outer surface with a central axis, an adjustable outer surface major diameter configured to be larger than the internal bore diameter of the tattoo machine apparatus externally threaded vise receiver, an adjustable outer surface minor diameter configured to be smaller than the internal bore diameter of the tattoo machine apparatus externally threaded vise receiver, a substantially cylindrical inner surface coaxial to the central axis of the conical outer surface with an adjustable inner diameter configured to accept a standard back stem style grip tube, a first bearing surface at the outer surface major diameter portion of the apparatus, a second bearing surface at the outer surface minor diameter portion of the apparatus, where the apparatus is coaxially inserted into the internal bore of the tattoo machine apparatus externally threaded vise receiver so that the first bearing surface protrudes outside of the tattoo machine apparatus externally threaded vise receiver.

The vise knob apparatus is substantially cylindrical, with a first end, an outer surface having a one or more grip features, a second end distal to the first end, a central bore extending from the first end to the second end defining an inner surface, where a portion of the inner surface extending inward from the second end is threaded such that the thread is configured to mate with the thread of the tattoo machine apparatus externally threaded vise receiver, and a portion of the inner surface extending inward from the first end has a one or more internal bearing surface configured to contact with the collet apparatus first bearing surface when the inner surface threads are engaged with the tattoo machine apparatus vise receiver external threads.

In light of the foregoing description, it should be recognized that embodiments in accordance with the present invention can be realized in numerous configurations contemplated to be within the scope and spirit of the claims. Additionally, the description above is intended by way of example only and is not intended to limit the present invention in any way, except as set forth in the claims.

What is claimed is:

1. A tattoo machine floating collet apparatus comprising:
a vise receiver, configured to receive a tattoo machine grip backstem, having a first end, a second end, and a body wall extending between the first end and second end defining an inner surface and an outer surface, where at least a portion of the outer surface extending from the first end towards the second end has an externally threaded portion;
a vise receiver knob, having an inner surface that is at least partially threaded so as to mechanically engage with the vise receiver externally threaded portion, an outer surface, and an aperture configured to receive a tattoo machine grip backstem; and
a vise collet, where the vise collet is configured to accept a tattoo machine grip backstem and be coaxially inserted into the vise receiver first end such that when the vise receiver knob is threaded onto the external threads of the vise receiver first end, the vise receiver knob engages with the vise collet, thereby causing the vise collet to tighten onto the tattoo machine grip backstem.

2. The apparatus of claim 1, where the vise collet is substantially conical.

3. The apparatus of claim 1, where the vise collet is substantially cylindrical.

4. The apparatus of claim 1, where the vise collet has a one or more mechanical features configured to prevent rotation of an inserted tattoo machine grip backstem.

5. The apparatus of claim 1, where the vise receiver knob inner surface includes a conical tapered area.

6. The apparatus of claim 1, where the vise receiver knob outer surface includes a one or more grip feature.

7. The apparatus of claim 1, where the vise receiver inner surface includes a conical taper area.

8. A collet vise system for accepting tattoo machine grips, the system comprising:
a tattoo machine apparatus having an externally threaded portion configured to accept a screw-on style adjustable grip with internal threading;
a collet apparatus having an outer surface, an inner surface with an adjustable inner diameter configured to accept a standard backstem style grip tube, where the collet apparatus is at least partially inserted into the tattoo machine apparatus externally threaded vise receiver; and
a vise knob apparatus, with a first end, an outer surface, a second end distal to the first end, a central bore extending from the first end to the second end defining an inner surface, where a portion of the inner surface extending inward from the second end is threaded such that the thread is configured to mate with the thread of the tattoo machine apparatus externally threaded vise receiver, and a portion of the inner surface extending inward from the first end has a one or more internal bearing surface configured to contact with the collet apparatus when the inner surface threads are engaged with the tattoo machine apparatus vise receiver external threads.

9. The apparatus of claim 8, where the collet apparatus is substantially conical.

10. The apparatus of claim 8, where the collet apparatus is substantially cylindrical.

11. The apparatus of claim 8, where the collet apparatus has a one or more mechanical features configured to prevent rotation of an inserted tattoo machine grip backstem.

12. The apparatus of claim 8, where the vise knob apparatus inner surface includes a conical tapered area.

13. The apparatus of claim 8, where the vise knob apparatus outer surface includes a one or more grip feature.

14. The apparatus of claim 8, where the tattoo machine apparatus externally threaded portion has an inner surface that includes a conical taper area.
